SEQUENCE LISTING (4 sequences)

SEQ ID NO: 1 - DNA, 19 bp, Artificial Sequence
  PI3 Forward Primer
ggcagctgtcacgggagtt

SEQ ID NO: 2 - DNA, 23 bp, Artificial Sequence
  PI3 Reverse Primer
cctgggcagtcagtatctttcaa

SEQ ID NO: 3 - DNA, 20 bp, Artificial Sequence
  CD14 Forward Primer
gggttcacagaggagggaac

SEQ ID NO: 4 - DNA, 18 bp, Artificial Sequence
  CD14 Reverse Primer
cgcgctccatggtcgata

(19) United States
(12) Patent Application Publication
(10) Pub. No.: US 2016/0319372 A1
(43) Pub. Date: Nov. 3, 2016

(54) METHOD FOR PREDICTING THE SURVIVAL STATUS AND PROGNOSIS OF ESOPHAGEAL CANCER AND A KIT THEREOF

(71) Applicant: KAOHSIUNG MEDICAL UNIVERSITY, Kaohsiung City (TW)

(72) Inventors: Ming-Tsang ; Wu, Kaohsiung City (TW); Deng-Chyang Wu, Kaohsiung City (TW); Hung-Ju Su, Kaohsiung (TW); Chah-Hwa Chou, Kaohsiung (TW); Jie-Len Huang, Kaohsiung (TW); Yu-Kuei Chen, Kaohsiung (TW); Chun-Chieh Wu, Kaohsiung (TW)

(73) Assignee: KAOHSIUNG MEDICAL UNIVERSITY, Kaohsiung City (TW)

(21) Appl. No.: 15/216,719

(22) Filed: Jul. 22, 2016

Related U.S. Application Data
(63) Continuation-in-part of application No. 13/426,485, filed on Mar. 21, 2012, now abandoned.

(30) Foreign Application Priority Data
Oct. 17, 2011 (TW) 100137548

(51) Int. Cl. C12Q 1/68; G01N 33/574

(52) U.S. Cl. CPC C12Q 1/6886; G01N 33/57407; C12Q 2600/158; C12Q 2600/118; G01N 2333/811; G01N 2333/70596

(57) ABSTRACT

The present invention provides a method for determining a survival rate and prognosis state for a patient having esophageal carcinoma, comprising: (a) providing a tissue sample from the patient; (b) measuring a expression level of a target gene in the tissue sample from the patient using reagents specific for the target gene product that are selected from the group consisting of probes, primers, antibodies, and antibody fragments, wherein the expression level is RNA expression, a protein expression at a cellular level or a tissue level; (c) comparing the expression level to a predetermined threshold indicative of a survival rate and prognosis state for the patient; and (d) providing a survival rate and prognosis state for the patient so that care for the patient is able to be determined in accordance with the survival rate and prognosis state, wherein the target gene is peptidase inhibitor 3 (PI3).

### CROSS REFERENCE

This application is a Continuation-in-part of the pending U.S. patent application Ser. No. 13/426,485 filed on Mar. 21, 2012, which is incorporated herein by reference in its entirety. This application also claim priority to Taiwanese Patent Application No. 100137548 filing on Oct. 17, 2011, which is incorporation herein by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the entire prior art of record and any search that the Office deems appropriate.

The Application contains a Sequence Listing in computer readable form. The computer readable form is incorporated herein by reference in its entirety.

### FIELD OF THE INVENTION

The present invention relates to a method and a kit for determining a survival rate and prognosis state for a patient having esophageal carcinoma based on the expression level of peptidase inhibitor 3 or (PI3) CD14 antigen.

### DESCRIPTION OF PRIOR ART

Esophageal carcinoma is one of the deadliest cancers with highly aggressive potency, ranking as the sixth most common cancer. The 5-year overall survival rate is less than 15%. The incidence is gradually increasing in the world every year. The esophageal carcinoma has multiple haplotypes, the tumor of esophageal usually causes dysphagia, pain and uncomfortable; in addition, patients need to be diagnosed by histology. Some small and non-transferred cancer can be treated with surgery; however, the strong invasive cancer must be treated by a medical therapy, a radio therapy or the combination thereof. The prognosis state of the esophageal carcinoma is determined that based on different degree of disease syndrome. Difficulty swallowing is a major symptom for a majority of the esophageal carcinoma patients, and it is possible to cause pain while wallowing. The liquid and the soft food usually can be accepted, however, the hard and solid food (e.g. bread or meat) is difficult for swallowing. An expression of weight loss is caused by a combination of the malnutrition and cancer. The common symptom is pain, particularly burning-like pain, which can be aching, associated with acute swallowing or throbbing; moreover, the cancerous swelling is possible to disturb normal stomach peristalsis, and then cause nausea, vomiting and regurgitation. A surface of tumor may easily crack and hemorrhage, clinical manifestation is hematemesis. The esophageal carcinoma in the later period may cause the caval syndrome. The other syndrome is fistula between the esophagus and the trachea. The foreign matter is through fistula into lung to cause cough, fever or pulmonary aspiration. In addition, the esophageal carcinoma of remote transfer can induce other symptom in the transfer site, for example, the liver transfer may cause jaundice and ascites. The lung transfer may cause shortness of breath and pleural effusion.

Thus, inventing a new method or finding a new gene for predicting a survival rate and prognosis state of esophageal carcinoma patients is very important.

The prior art for predicting a survival rate and prognosis state of esophageal carcinoma patients is detecting a specific sequence or related protein of esophageal carcinoma by a probe or an antibody, or determining that whether the cancer cell can be killed by chemotherapy by Single Nucleotide Polymorphism (SNP) test. However, it is inaccurate that an uncertain detecting target in prior arts and the procedure is too complex.

Furthermore, US patent application No. 2009/0208514 A1 disclosed a method that detecting the prognosis of esophageal carcinoma by gene DKK1, but it may be not accurate that only detecting one gene.

The present invention provides a new method and a kit for determining a survival rate and prognosis state for a patient having esophageal carcinoma by two detecting targets that can improve the accuracy and simplify the procedures.

### SUMMARY OF THE INVENTION

The present invention provides a method for determining a survival rate and prognosis state for a patient having esophageal carcinoma, comprising:

(a) providing a tissue sample from the patient;

(b) measuring a expression level of a target gene in the tissue sample from the patient using reagents specific for the target gene product that are selected from the group consisting of probes, primers, antibodies, and antibody fragments, wherein the expression level is RNA expression, a protein expression at a cellular level or a tissue level;

(c) comparing the expression level to a predetermined threshold indicative of a survival rate and prognosis state for the patient; and

(d) providing a survival rate and prognosis state for the patient so that care for the patient is able to be determined in accordance with the survival rate and prognosis state, wherein the target gene is peptidase inhibitor 3 (PI3).

The present invention provides a method for predicting a survival rate and prognosis state of esophageal carcinoma patients, comprising:

(a) providing a tissue sample from a case subject;

(b) examining a expression level of CD14 antigen (CD14) or peptidase inhibitor 3 (PI3) in the case subject, comprising a RNA expression, a protein expression at a cellular level or a tissue level; and

(c) comparing the expression level of CD14 antigen or peptidase inhibitor 3 (PI3) with a control subject;

wherein the expression level of CD14 is higher than the control subject which means with a better survival rate and a better prognosis, and the expression level of PI3 is higher than the control subject, which means with a worse survival rate and a worse prognosis.

The present invention further provides a kit comprising: a pair of primers for amplifying CD14 antigen or peptidase inhibitor 3 (PI3); wherein the pair of primers for amplifying CD14 consisting of SEQ ID No: 1 and SEQ ID No. 2; and the pair of primers for amplifying PI3 consisting of SEQ ID No: 3 and SEQ ID No. 4.

The present invention also provides a kit comprising a microarray chip for predicting a survival rate and prognosis state of esophageal carcinoma patients, wherein a surface of the microarray chip is coated with a nucleotide sequence that recognizes RNA of gene CD14 or two nucleotide sequences that recognize RNA of gene CD14 and RNA of gene peptidase inhibitor 3 (PI3); wherein the nucleotide sequence that coated on the surface of the microarray chip and recognizes RNA of gene PI3 consists of the nucleotide sequence set forth in SEQ ID No: 1 and SEQ ID No. 2; and the nucleotide sequence that recognizes RNA of gene CD14 consists of the nucleotide sequence set forth in SEQ ID No: 3 and SEQ ID No. 4.

### DETAILED DESCRIPTION OF THE INVENTION

The present invention provides a method for determining a survival rate and prognosis state for a patient having esophageal carcinoma, comprising:

(a) providing a tissue sample from the patient;

(b) measuring a expression level of a target gene in the tissue sample from the patient using reagents specific for the target gene product that are selected from the group consisting of probes, primers, antibodies, and antibody fragments, wherein the expression level is RNA expression, a protein expression at a cellular level or a tissue level;

(c) comparing the expression level to a predetermined threshold indicative of a survival rate and prognosis state for the patient; and

(d) providing a survival rate and prognosis state for the patient so that care for the patient is able to be determined in accordance with the survival rate and prognosis state, wherein the target gene is peptidase inhibitor 3 (PI3).

In one embodiment, the target gene further comprises CD14 or CD14 antigen.

In one embodiment, the predetermined threshold is an average expression level from other patients having esophageal carcinoma.

In one embodiment, the survival rate and prognosis state for the patient is better when the expression level of PI3 is lower than the predetermined threshold and the survival rate and prognosis state for the patient is worse when the expression level of PI3 is higher than the predetermined threshold.

In one embodiment, the survival rate and prognosis state for the patient is better when the expression level of CD14 is higher than the predetermined threshold and the survival rate and prognosis state for the patient is worse when the expression level of CD14 is lower than the predetermined threshold.

In one embodiment, the RNA expression is measured by real-time PCR using primer pair specific for the target gene product.

In one embodiment, the primer pair specific for PI3 gene product is SEQ ID No: 1 and SEQ ID No. 2, the primer pair specific for CD14 gene product is SEQ ID No: 3 and SEQ ID No. 4

The present invention provides a method for predicting a survival rate and prognosis state of esophageal carcinoma patients, comprising:
(a) providing a tissue sample from a case subject;
(b) examining a expression level of CD14 antigen (CD14) or peptidase inhibitor 3 (PI3) in the case subject, comprising a RNA expression, a protein expression at a cellular level or a tissue level; and
(c) comparing the expression level of CD14 antigen or peptidase inhibitor 3 (PI3) with a control subject;
   wherein the expression level of CD14 is higher than the control subject which means with a better survival rate and a better prognosis, and the expression level of PI3 is higher than the control subject, which means with a worse survival rate and a worse prognosis.

In the method of the present invention, the real-time PCR or a DNA microarray chip is one of the ways to examine the RNA expression level, the protein microarray chip, Western blotting or immunohistochemistry (IHC) is one of the ways to examine the protein expression at cellular level or tissue level.

In one embodiment, the RNA expression is examined by the real-time PCR or the DNA microarray chip in the present invention.

In one embodiment, the protein expression at cellular level is examined by a Western blotting, and the tissue protein expression of CD14 or PI3 from the case subject and the control subject is examined by immunohistochemistry (IHC).

The present invention further provides an isolated DNA primer consisting of the nucleotide sequences in SEQ ID No: 1 and SEQ ID No. 2.

The present invention further provides an isolated DNA primer consisting of the nucleotide sequences in SEQ ID No. 3 and SEQ ID No. 4.

The present invention provides a kit for predicting a survival rate and prognosis state of esophageal carcinoma patients comprising:

a pair of primers for amplifying CD14 antigen or peptidase inhibitor 3 (PI3);

wherein the pair of primers for amplifying CD14 consisting of SEQ ID No: 1 and SEQ ID No. 2; and

the pair of primers for amplifying PI3 consisting of SEQ ID No: 3 and SEQ ID No. 4.

In one embodiment, the kit further comprises means for examining a RNA expression level of CD14 antigen or peptidase inhibitor 3 (PI3) from a case subject by a real-time PCR.

The present invention further provides a microarray chip for predicting a survival rate and prognosis state of esophageal carcinoma patients, wherein the surface of the microarray chip is coated with either nucleotide sequence or antibody that recognize the RNA or protein product of gene CD14 or PI3.

The present invention further provides a kit comprising a microarray chip for predicting a survival rate and prognosis state of esophageal carcinoma patients, wherein a surface of the microarray chip is coated with a nucleotide sequence that recognizes RNA of gene CD14 or two nucleotide sequences that recognize respectively RNA of gene CD 14 and RNA of gene peptidase inhibitor 3 (PI3).

In one embodiment of the present invention, the nucleotide sequence that coated on the surface of the microarray chip and recognizes RNA of gene CD 14 consists of the nucleotide sequence set forth in SEQ ID NO: 1 and SEQ ID NO: 2; and the nucleotide sequence that coated on the surface of the microarray chip and recognizes RNA of gene peptidase inhibitor 3 (PI3) consists of the nucleotide sequence set forth in SEQ ID NO: 3 and SEQ ID NO: 4.

### BRIEF DESCRIPTION OF THE DRAWINGS

The accompanying drawings illustrate preferred embodiments of the present invention as well as other information pertinent to the disclosure, in which:

FIG. 1 shows the functional expression of peptidase inhibitor 3 (PI3) in CE81T2 and CE81T2-4 cell lines. (A) PI3 intensity in Human Whole Genome OneArray™. (B) PI3 mRNA expression level is examined by real-time PCR (P<0.05). (C) PI3 protein expression level is examined by Western blotting (total protein loading 40 μg). (D) PI3 protein level is examined in paraffin-embedded cell block (4 μm thick) by IHC staining (arrows).

FIG. 2 shows the functional expression of CD14 antigen (CD14) in CE81T2 and CE81T2-4 cell lines. (A) CD14 intensity in Human Whole Genome OneArray™. (B) CD14 mRNA expression level is examined by real-time PCR (P<0.05). (C) CD14 protein expression level is examined by Western blotting (total protein loading 40 μg). (D) CD14 protein expression level is examined in paraffin-embedded cell block (4 μm thick) by IHC staining (arrows).

FIG. 3 shows the distribution of staining scores of PI3 and CD14 by IHC staining (200×) in obtaining esophageal cancer tissue samples.

FIG. 4 shows the stained intensity of PI3 and CD14 expression levels in two groups of representative ESCC (esophageal squamous cell carcinoma) patients with worse or better prognosis by IHC stained method in one tissue array chip slide. (A) Layout of tissue array chip. One tissue array slide with 10 case sets. There are two case sets in one row and triplicate cores per case, and one black dot in left-upper part represents “Mark”. (B) Functional expression of PI3 and CD14 (200×).

### EXAMPLES

The examples below are non-limiting and are merely representative of various aspects and features of the present invention.

The present invention observed and selected the malignant daughter cell with the different invasion ability by Transwell invasion chamber assay from a Taiwanese esophageal squamous cell carcinoma (ESCC) of cell line (CE81T/VGH). Then the present invention used the whole genome microarray chip (Human OneArray™, HOA) to compare the parent cell line (CE81T1/CE81T2-4) and the malignant daughter cell (CE81T2/CE81T2-4). According to (1) the enrichment analysis data indicated the genes that associated with cell adhesion, cytoskeletons reconstruction, and cell membrane rebuild, or the genes related to formation or progress of tumor and cancer are supported by the literatures; (2) the genes showed medium to high expression in original data; (3) products of the genes were secreted to or located at extracellular, cell membrane or cytoplasm.

The present invention identified 13 candidate genes potentially with encoded extracellular or membrane proteins related to their invasion ability, showed in Table 1; wherein the expression level of five genes of these candidate genes were up-regulated and the others of these candidate genes were down-regulated.

In addition, after the present invention studied the functional expressions of 13 candidate genes in vitro, and found peptidase inhibitor 3(PI3) and CD14 antigen (CD14) had high relativity to the survival status and prognosis of esophageal cancer.

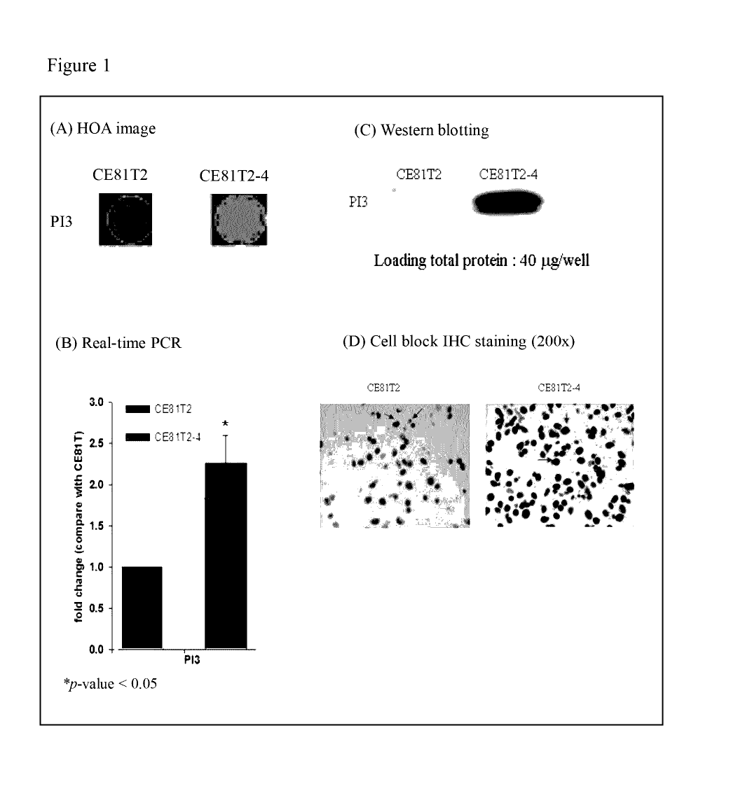

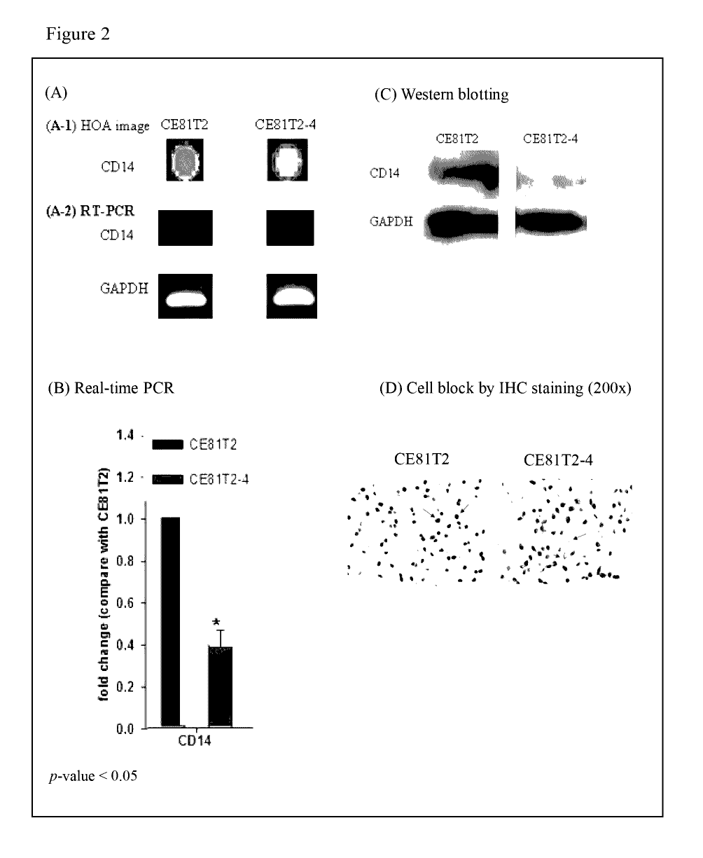

The present invention used CE81T2 and CE81T2-4 of the malignant daughter cell to observe the expression level of RNA and protein of PI3 and CD14, wherein CE81T2-4 was more malignant than CE81T2. RNA expression level of PI3 and CD14 was examined by real-time polymerase chain reaction (RT-PCR) or DNA chip, and protein expression level was examined by Western-blot or immunohistochemistry (IHC). The results were showed in FIG. 1 and FIG. 2.

TABLE 1
The 13 candidate genes were selected from esophageal squamous cell
carcinoma (ESCC) cell lines and their daughter cells by oligonucleotide
chip with human membrane protein/secreted protein.

| | | | Cellular |
|---|---|---|---|
| | Gene Symbol | Gene Name | location |
| Up- | | | |
| regulated | — | | |
| 1 | PI3 | Peptidase inhibitor 3 | Extracellular |
| 2 | CXCL14 | Chemokine (C—X—C motif) | Extracellular |
| | | ligand 1 | |
| 3 | GJA1 | Gap junction protein, | Membrane |
| | | alpha 1, 43 kDa | |
| 4 | MMP1 | Matrix metalloproteinase 1 | Extracellular |
| 5 | NTS | Neurotensin | Extracellular |
| Down- | | | |
| regulated | — | | |
| 1 | CD14 | CD14 antigen | Membrane |
| 2 | MMP9 | Matrix metalloproteinase 9 | Extracellular |
| 3 | FN1 | Fibronectin 1 | Extracellular |
| 4 | SCNN1A | Sodium channel, | Membrane |
| | | nonvoltage-gated 1 alpha | |
| 5 | SPP1 | Secreted phosphoprotein 1 | Extracellular |
| | | (osteopontin) | |
| 6 | TNFRSF11B | Tumor necrosis factor | Membrane |
| | | receptor superfamily, | |
| | | member 11b | |
| | | (osteoprotegerin) | |
| 7 | MUC1 | Mucin 1, transmembrane | Membrane |
| 8 | MUC4 | Mucin 4, transmembrane | Membrane |

### Example 1

### Detecting the RNA Expression of PI3 and CD14 by Real-Time PCR

As showed in FIG. 1, PI3 gene was strongly expressed in cell line ESCC CE81T2-4, but PI3 gene was not or weakly expressed in CE81T2. In addition, as showed in FIG. 2(A-1), the signal expression of CD14 was higher in CE81T2 than in CE81T2-4. Then the present invention used real-time PCR to quantify RNA expression levels of PI3 and CD14 in CE81T2 and CE81T2-4.

Total RNA from cell lines was extracted, and then reverse transcribed to cDNA. After reverse transcription, 300 ng cDNA was mixed with IX SYBR Green Master Mix (Roche Cat; NO. 03 531 295 001) and 10 mM primers (the primer sequences are showed in Table 2 and SEQ ID NO.1˜4), and the real-time PCR was performed. The present invention evaluated PI3, CD14 and glyceraldehydes-3-phosphate dehydrogenase (GADPH) expressions by real-time PCR.

Quantitative real-time PCR was performed using an ABI STEP-ONE REAL-TIME PCR SYSTEM® (Applied Biosystems).

Amplification conditions were as follows: initial denaturation temperature at 95° C. for 10 minutes, followed by 40 complete cycles of denaturation temperature at 94° C. for 15 seconds and annealing temperature at 60° C. for 1 minute. The expression levels of PI3 and CD14 in CE81T2 and CE81T2-4 were showed in FIG. 1B and FIG. 2 (A-2 and B).

FIG. 1B showed, PI3 of the RNA expression level in CE81T2-4 were 2 times as in CE81T2, and FIG. 2 (A-2 and B) showed CD14 of the RNA expression level in CE81T2 was higher than in CE81T2-4. In addition, as showed in FIG. 2B, CD14 expression level at the RNA level in CE81T2-4 was about 0.4 times than in CE81T2.

TABLE 2
Primer Sequences

| | | | Primer size |
|---|---|---|---|
| Gene | Primer sequences | | (bp) |
| P13 | Forward: | 5′-GGC AGC TGT CAC GGG AGT T-3′ | 237 |
| | Reverse: | 5′-CCT GGG CAG TCA GTA TCT TTC AA-3′ | |
| CD14 | Forward: | 5′-GGG TTC ACA GAG GAG GGA AC-3′ | 91 |
| | Reverse: | 5′-CGC GCT CCA TGG TCG ATA-3′ | |
| GAPDH | Forward: | 5′-GCA CCG TCA AGG CTG AGA AC-3′ | 142 |
| | Reverse: | 5′-ATG GTG GTG AAG ACG CCA GT-3′ | |

### Example 2

### Detecting the Protein Level of PI3 and CD14 by Western Blotting

The present invention used Western blotting to examine PI3 and CD 14 protein expression levels in CE81T2 and CE81T2-4. The experimental method as following: Cells for detecting CD14 protein were collected and extracted by using lysis buffer (150 mmol/L NaCl, 0.1% sodium dodecyl sulfate, 10 mmol/L EDTA, and 1% NP40, 1× protease inhibitor (Roche) and 50 mmol/L Tris-HCl (pH 7.5); and cells for detecting PI3 protein were collected and extracted by using the trichloroacetic acid (TCA) precipitation method.

After the total protein was extracted by above methods, the present invention used BCA protein assay (Pierce) to determine the protein concentration. Protein samples were mixed 1×SDS protein loading buffer (Invitrogen) and heated at 70° C. for 10 minutes. After that, equal amounts of protein (40 μg) were separated on 4-12% gradient acrylamide gel (Invitrogen). After electrophoresis, the protein samples were transferred to a PVDF membrane (Pall). Membranes were blocked with Tween 20 (TBS-T) containing 5% (w/v) non-fat dry milk, and then probed with primary antibodies. Western analysis was performed using the following antibodies: anti-CD 14 rabbit polyclonal antibody (sc-58954, SantaCruz) and anti-PI3 rabbit polyclonal antibody (sc-20637, SantaCruz). After incubation, membranes were incubated with horseradish peroxidase (HRP)-conjugated secondary antibodies (1:5000).

The present invention used a chemiluminescence detection system (Millipore) to detect signal of the membranes, the membranes were washed, followed by incubation in substrate, and exposed to film. The same membrane was re-probed with glyceraldehydes-3-phosphate dehydrogenase (GAPDH) polyclonal antibody (Ab FRONTIER) as an internal control. To re-probe with GAPDH, the membrane was incubated with stripping buffer (100 mM β-mercaptoethanol, 2% SDS, and 62.5 mM Tris-HCl) at 55° C. for 20 min and washed with distilled water for further study.

The results were showed in FIG. 1C and FIG. 2C, the PI3 protein was highly expressed in CE81T2-4, but was not detected in CE81T2. In addition, as FIG. 2C showed, CD 14 expression level in CE81T2 was higher than in CE81T2-4.

### Example 3

### Detecting the Protein Expression of PI3 and CD14 by the Immunohistochemistry (IHC)

The present invention provided a method of detecting the protein expression in CE81T2 and CE81T2-4 by immunohistochemistry (IHC). These results were similar as mentioned above.

The present invention used same antibodies to perform IHC and Western blotting (FIG. 1D and FIG. 2D), that was, anti-CD14 rabbit polyclonal antibody (sc-58954, SantaCruz) and anti-PI3 rabbit polyclonal antibody (sc-20637, SantaCruz). As FIG. 1D showed, PI3 protein expressed was higher in CE81T2-4, and FIG. 2D showed that CD14 protein level in CE81T2-4 was lower than CE81T2.

Summarizing the results, as FIG. 1D and FIG. 2D showed, PI3 expression level was high and CD14 expression level was low in CE81T2-4 cell line. However, PI3 expression level was low but CD14 expressed was high in CE81T2 cell line. As the description above, due to CE81T2-4 was more malignant than CE81T2, CE81T2-4 belonged to low survival rate and prognosis, however CE81T2 represented high survival rate and prognosis

The present invention selected randomly the cancer tissue samples fixed in FFPE (Formalin-Fixed and Parrffin-Embedded) of two groups of esophageal carcinoma patients to make pathological sections, and two groups were classified by the survival mouths and stage IIIA (AJCC, 2010). The patients were classified to better prognosis (the average survival was about 25 months) and worse prognosis (the average survival was about 7 months), and each group had three male patients (the classification was done by a pathology doctor who didn't know the survival months of the patients).

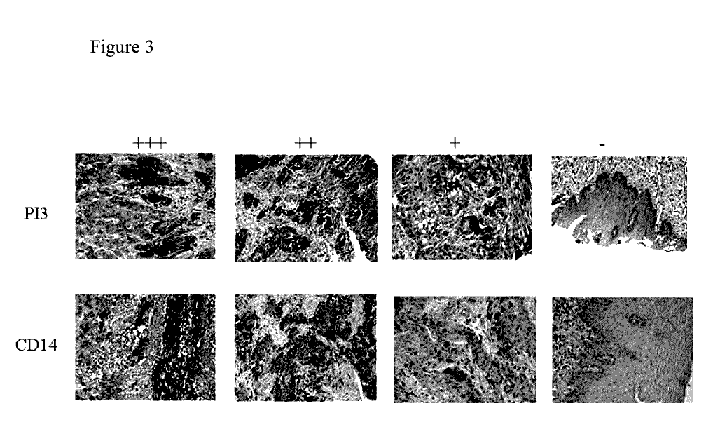

FIG. 3 showed the distribution of staining scores of PI3 and CD14 by IHC staining in obtaining esophageal cancer tissue samples. That was to determine the protein expression levels of PI3 and CD14 in esophageal squamous cell carcinoma (ESCC).

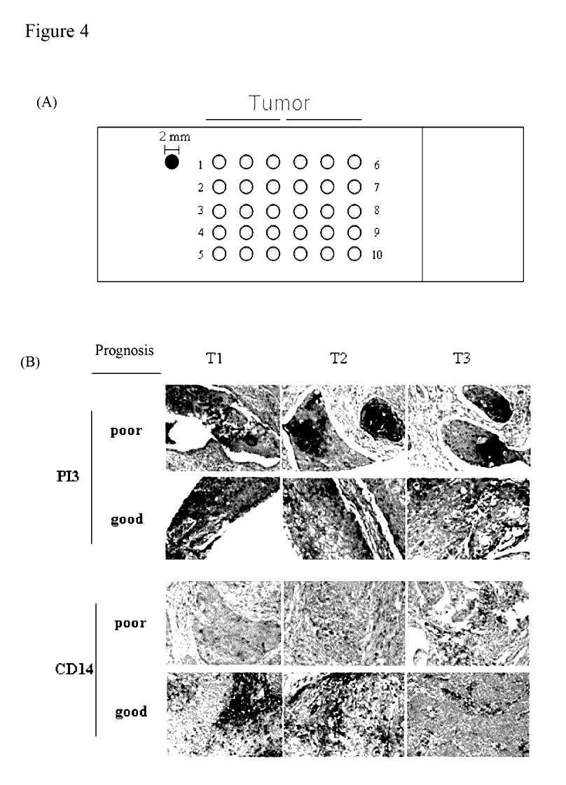

FIG. 4A showed. One tissue array slide contained 10 case sets. There are two case sets in one row, and triplicate cores per case. One black dot in left-upper part represented “Mark”. The IHC staining results were showed in FIG. 4B, wherein PI3 had high protein expression level in worse prognosis group and low protein expression level in better prognosis group; and CD14 had high protein expression level in better prognosis group and low protein expression level in worse prognosis group.

The present invention used IHC comparative figure of FIG. 3 to compare with the results of FIG. 4, and dealt with Table 3, wherein the PI3 protein expression was low level and the CD14 protein expression was high level in the tissue sample of the patients who had high survival rate and good prognosis, whereas the PI3 protein expression was high level and the CD14 protein expression was low level in the tissue sample of the patients who had low survival rate and poor prognosis.

As the description above, the above examination for the expression level of PI3 and CD14 in RNA or protein level could used to predict the survival rate and the prognosis state of esophageal carcinoma patients.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in protein structure without affecting the biological action in kind or amount. All such modifications are intended to be included within the scope of the appended claims.

TABLE 3
Sample survey form for esophageal cancer patent

| | | | | | | | | Combination | | | | |
|---|---|---|---|---|---|---|---|---|---|---|---|---|
| | | | | | | | | of medical and | | | Gene expression | |
| | | | | | | | Differentiation | radiation | | | intensity | |
| Number | Age | Tumor site | T | N | M | Stage | ability | therapy | Surgery | Survival time | PI3 | CD14 |

Good group of prognosis

| | | | | | | | | | | | | |
|---|---|---|---|---|---|---|---|---|---|---|---|---|
| 20521 | 65 | Middle ⅓ | 2 | 0 | 0 | IIA | High | Y | Y | 23 | + | ++ |
| 20971 | 66 | Upper ⅓ | 3 | 0 | 0 | IIA | Low | Y | Y | 29 | + | +++ |
| 21411 | 45 | Middle ⅓ | 2 | 0 | 0 | IIA | Low | N | Y | 25 | − | +++ |

Poor group of prognosis

| | | | | | | | | | | | | |
|---|---|---|---|---|---|---|---|---|---|---|---|---|
| 20591 | 66 | Middle ⅓ | 2 | 0 | 0 | IIA | moderate | Y | Y | 10 | +++ | + |
| 21201 | 67 | Middle ⅓ | 3 | 0 | 0 | IIA | moderate | N | Y | 5 | +++ | + |
| 21621 | 60 | Low ⅓ | 3 | 0 | 0 | IIA | moderate | N | Y | 7 | ++ | + |

T2: tumor invades muscularis propria;
T3: tumor invades adventitia;
M0: no distant metastasis
CCRT: Concurrent chemoradiation therapy

What is claimed is:

1. A method for determining a survival rate and prognosis state for a patient having esophageal carcinoma, comprising:
(a) providing a tissue sample from the patient;
(b) measuring a expression level of a target gene in the tissue sample from the patient using reagents specific for the target gene product that are selected from the group consisting of probes, primers, antibodies, and antibody fragments, wherein the expression level is RNA expression, a protein expression at a cellular level or a tissue level;
(c) comparing the expression level to a predetermined threshold indicative of a survival rate and prognosis state for the patient; and
(d) providing a survival rate and prognosis state for the patient so that care for the patient is able to be determined in accordance with the survival rate and prognosis state,
wherein the target gene is peptidase inhibitor 3 (PI3).

2. The method of claim 1, wherein the target gene further comprises CD14 or CD14 antigen.

3. The method of claim 1, wherein the predetermined threshold is an average expression level from other patients having esophageal carcinoma.

4. The method of claim 1, wherein the survival rate and prognosis state for the patient is better when the expression level of PI3 is lower than the predetermined threshold and the survival rate and prognosis state for the patient is worse when the expression level of PI3 is higher than the predetermined threshold.

5. The method of claim 2, wherein the survival rate and prognosis state for the patient is better when the expression level of CD14 is higher than the predetermined threshold and the survival rate and prognosis state for the patient is worse when the expression level of CD14 is lower than the predetermined threshold.

6. The method of claim 1, wherein the RNA expression is measured by real-time PCR using primer pair specific for the target gene product.

7. The method of claim 1, wherein the RNA expression is measured by a microarray chip.

8. The method of claim 1, wherein the protein expression is measured by a Western blotting.

9. The method of claim 1, wherein the protein expression is measured by immunohistochemistry (IHC).

10. The method of claim 6, wherein the primer pair specific for PI3 gene product is SEQ ID No: 1 and SEQ ID No. 2.

11. The method of claim 6, wherein the primer pair specific for CD14 gene product is SEQ ID No: 3 and SEQ ID No. 4.
